(19) United States
(12) Patent Application Publication
(10) Pub. No.: US 2010/0307526 A1
(43) Pub. Date: Dec. 9, 2010

(54) Composition and method for hair straightening and curling

(76) Inventors: Robert Saute, Los Angeles, CA (US); Steve Saute, La Canada Flintridge, CA (US)

Correspondence Address: SANDRA LEE LIPKIN A LAW CORPORATION, P.O.Box 3518, VENTURA, CA, 93006, US

(21) Appl. No.: 12/802,035

(22) Filed: May 28, 2010

Related U.S. Application Data
(60) Provisional application No. 61/217,796, filed on Jun. 3, 2009.

(51) Int. Cl. A45D 7/06; A61K 8/88; A61Q 5/06

(52) U.S. Cl. 132/206; 424/70.17

(57) ABSTRACT

A composition and method of use therefore that allows for permanent straightening or curling of human hair that is not irritating to the skin, that allows for the immediate shampooing of hair thereafter and that provides for re-treatment of the hair without additional damage. The composition primarily uses dimethyl sulfone (MSM) and high temperatures to break and reform disulfide bonds as a means to allow for the penetration of low molecular weight proteins into the shaft of the hair. When curling the hair, it is understood that devices that will stretch hairs into curls at the temperatures expressed in the scope of the patent will produce a permanent curled hair. A test curl will indicate the heat and temperature required to permanently curl the hair being treated.

### REFERENCE TO PRIOR APPLICATION

This application claims the priority of provisional application 61/217,796, filed Jun. 3, 2009 entitled COMPOSITION AND METHOD FOR HAIR STRAIGHTENING by Robert Saute and Steve Saute.

### BACKGROUND OF THE INVENTION

1. Field of the Invention

The product invention related generally to the field of the hair manipulation products and systems, and particularly to a process and series of compounds that permanently straighten and/or curl hair.

2. Description of the Prior Art

The art of curling hair dates back as far as ancient Egypt, when the “mud method” was used. Wooden sticks used in the “mud method” were latter replaced by metal rods, and as time progressed the rods were heated during the process. This use of heating rods was the foundation for the use of curling tongs that were still popular in the 1930s. The first permanent waving method introduced into the United States originated in France in 1884. This method included the use of concentrated seawater and heat.

All previous methods were abandoned when Charles Nessler devised a process for softening the hair using borax in 1906. Evans and McDonough introduced a method using mercaptans, first as depilatories and as cold waving agents. The substituted mercaptans were found preferable in curling the hair because they are more efficient and have a less objectionable odor.

Various modifications to the use of the mercaptans products have been developed to improve the curling power and to reduce the damage done to the hair during the curling process. The most popular waving preparations have an alkaline pH in the range of 8.0 to 9.0. Acid waves, using derivatives of thioglycolic acid are simple amide salts and esters. Amides and esters of thioglycolic acid are potential sensitizers. These products are of little commercial importance due to their instability and irritation potential. Sulfite containing products can be used in tepid waving and found in hair straightening in the presence of large amounts of urea and isopropyl alcohol.

Tepid waving, a form of heat waving has been used to increase reaction time between sulfites and hair. The temperatures used are moderate and well tolerated by consumers under a hair dryer. Steam flat irons and dry flat irons are used to straighten hair. When the tepid method is used the strength of conventional heat waving preparations is increased, while it is decreased in the cold wave products. Temperatures in the range of 20 C to as high as 80 C have been used in these procedures.

The physical process of permanently waving or straightening hair is cause by a change in the molecular configuration of the keratin molecule, giving the hair a lasting curl that cannot be changed by physical means. This change in configuration can be accomplished by the use of high temperatures, or by chemicals such as alkalis and certain sulfur compounds adjusted to a proper pH, in bases having a dissociation constant of less than 5×10^{-3.5}. Water curls and temporary sets differ from permanent waves in that the curls are formed when unstable cross linkages are present, and tend to return to their normal configuration when moistened. The hair in its normal state is made up of regularly folded polypeptide chains with cystine, hydrogen and polar or salt linkages.

In order to permanently curl or straighten the hair, one must break the disulfide bonds. Before the chemical can break these disulfide bonds it must penetrate the cuticle of the hair. Both permanent waving of the hair and straightening of the hair have been popular ways of modifying hairstyles. People with unmanageable tight curly hair frequently have their hair straightened. Straightening the hair has been accomplished by using highly viscous alkaline preparations; the high viscosity helps to keep the preparation off the scalp. The most popular preparations are formulated with sodium hydroxide or potassium hydroxide. The pHs of these preparations range from eight to twelve.

All prior art products either permanently straighten or curl the hair using ingredients that are irritating to the skin. Consequently, manufacturers caution people who use these products to use protective gloves to apply the creams or solutions to the hair. They recommend protecting the skin of the person receiving the straightening or curling service as much as possible.

In general the alkaline products are left on the hair for a specified length of time. A test curl is taken to determine if the time of contact is sufficient to produce a satisfactory curl. When straightening the hair, the product is placed on the hair and stretched using a styling tool or brush. Large curlers are used to obtain wavy hair. In both curling and straightening processes, there is a second step required to reform the disulfide bonds in the hair. This is necessary to make reconfiguration permanent.

The shortcomings in the prior art are many fold. The primary drawback is the potential damage to the skin of the person receiving the treatment based on the high pH of the solutions used. Additionally, the processes provide a noxious smell that takes days or weeks to leave the hair. Also, universally, the hair is left in a damaged state and weaker than prior to treatment. Accordingly, when the treated hair grows out, in order to maintain the curl or straightened state of the hair, only the newly grown hair can be treated without further damaging the hair. Another drawback to prior art methods is that the hair cannot be shampooed for several days after the procedure because the achieved result will be negated by shampooing in the days following.

It is the object of the present invention to overcome all of the drawbacks of the prior art by providing a composition of products and a process for use thereof that achieves the desired ends without any of the stated drawbacks.

It is an object of the invention to provide a product and process for straightening or curling hair that does not damage the skin of the person receiving the treatment based on the high pH of the solutions used.

It is an object of the invention to provide a product and process for straightening or curling the hair that does not leave a noxious smell in the hair of the person.

It is an object of the invention to provide a product and process for straightening or curling the hair that does not leave the hair in a damaged state and that actually may leave the hair in better physical shape than prior to the treatment.

It is an object of the invention to provide a product and process for straightening or curling hair that does not incur damage with multiple treatments.

It is an object of the invention to provide a product and process for straightening or curling hair that does not become undermined by the immediate shampooing of the hair after treatment.

### SUMMARY OF THE INVENTION

The preferred embodiment of the present invention teaches a one part solution for the straightening or curling of hair, said solution comprising comprising 57-92% water, 3.5-8% PEG-8, 2-10% Urea, 0.5-3.0% Polysorbate 20, 0.005-0.5% Methylisothiazolinone, 0.1-0.5% Hydrolyzed Corn Protein, 0.1-0.5% Oat Amino Acids, 0.1-0.5% Hydrolyzed Soy Protein, .02-0.1% Disodium EDTA, 0.05-0.50% Hydrolyzed Abalone Protein, 0.5-3% Ethoxydiglycol, 0.1-0.5% Sodium Polystyrene Sulfonate, 0.2-10 Dimethyl Sulfone, 0.25-1% Panthenol, 0.1-0.75 Triethyl Citrate, 0.05-0.25 Phylantriol, 0.1-0.75 Polyacrylamidomethylpropane Sulfonic Acid, applying said solution on the hair; applying to the hair soaked with said solution a hot iron set a temperature between 200 and 500 degrees Fahrenheit; with said hot iron, straighten the hair using tension and a comb between one and six times as dictated by the type of hair.

The above embodiment can be further modified by defining that the solution further comprises 0.5-1.5% fragrance.

The above embodiment can be further modified by defining that the solution further comprises 0.5-3.0% extracts of Althea Officinalis Root, Chamomilla Recutita (Matricaria) Flower, Citrus Aurantium Dulcis (Orange) Peel, Achillea Millefolium, Foeniculum Vulgare (Fennel) Fruit, Glycyrrhiza Glabra (Licorice) Rhizome/Root, Ginkgo Biloba Leaf.

### DETAILED DESCRIPTION OF A PREFERRED EMBODIMENT

The instant invention seeks to provide a new hair straightening and curling process and product that produces permanent reconfiguration of hair. One unique product is applied to the hair and heated with a dry or steam iron according to specific instructions. The hair can be shampooed immediately after the procedure, leaving a permanently curled or straightened hair in excellent condition. The treatment does not leave an obnoxious odor in the hair or release obnoxious odors while the hair is treated. A complete process can be accomplished in less than half the time it takes to accomplish the task with prior art processed and products.

There is one composition used in the process described herein. The products used in the process of rearranging the molecular structure of the hair to create either curly or straight hair are unique in that they are not alkaline and do not produce obnoxious odors. These products are not considered irritants or sensitizers. The application of this product does not require the use of rubber gloves or need to protect the skin with an ointment.

### Composition of Product

The product consists of several hydrolyzed proteins derived from land and sea. These hydrolyzed proteins consist of amino acids, peptides and polypeptides. The first product also consists of humectants, such as glycerin, sodium PCA, Dimethyl Sulfone (MSM) and urea; surfactants, such as those that are ionic and nonionic, sodium lauryl sulfate and polysorbates; lubricants, such as PEG-12, propylene glycol and butylenes glycol; fragrances, herbal extracts and preservatives.

The product can be made in one vessel by one skilled in the art of compounding, while controlling the stirring so as to avoid the entrapment of air therein.

Additional compatible additives to each product may be used for obtaining particular effects such as volatile solvents, color, sunscreens, etc, which are well know by those who are skilled in the art.

Once compounded, the product needs to be used following a very specific procedure in order to achieve the desired result with the desired benefits over the prior art. The composition of the product used in conjunction with the procedure defined herein provides excellent results in either straightening or curling the hair.

The procedure used to straighten the hair consists of shampooing the hair with a cleansing shampoo, then blow-drying the hair. The product is applied to the hair. The hair is then hot ironed while applying stress to the hair with a styling tool or brush. The ironing procedure is repeated several times while maintaining a suitable temperature with the iron.

### Formulation Example for Product 1

| Ingredient | % by Weight |
|---|---|
| Water | 83.02 |
| Dimethyl Sulfone | 8.0 |
| PEG-8 | 5.0 |
| Hydrolyzed Corn Protein 35% | 1.0 |
| Glycerin | 1.0 |
| Polysorbate 20 | 1.0 |
| Hydrolyzed Soy Protein | 0.5 |
| Hydrolyzed Abalone Protein | 0.1 |
| Triethyl Citrate | 0.1 |
| Sodium Polystyrene Sulfonate | 0.1 |
| Citric Acid | 0.1 |
| Disodium EDTA | 0.03 |
| Methylisothiazolinone | 0.05 |

Step One: Wash the hair with an appropriate shampoo for the hair type.

Step Two: Dry the hair thoroughly.

Step Three: Apply the product to the hair.

Step Four: Use a hot iron at a temperature at 410 F.

Step Five: Straighten the hair with the iron at 410 F using the tension and a comb.

Step Six: Repeat two to six times, depending on the hair type.

When curling the hair, it is understood that devices that will stretch hairs into curls at the temperatures expressed in the scope of the patent will produce a permanent curled hair. A test curl will indicate the heat and temperature required to permanently curl the hair being treated.

Use of this product is also possible for semi-permanent straightening. The steps involved in this method include the following:

Step One: Wash the hair with a clarifying shampoo.

Dry the hair completely.

Apply the above composition to the hair.

Dry the hair completely.

Straighten the hair with a flat iron at 410° F.

Repeat from one to eight times.

The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

What is claimed is:

1. A one part solution for the straightening or curling of hair, said solution comprising:
57-92% water;
3.5-8% PEG-8;
2-10% Urea;
0.5-3.0% Polysorbate 20;
0.005-0.5% Methylisothiazolinone;
0.1-0.5% Hydrolyzed Corn Protein;
0.1-0.5% Oat Amino Acids;
0.1-0.5% Hydrolyzed Soy Protein;
0.02-0.1% Disodium EDTA;
0.05-0.50% Hydrolyzed Abalone Protein;
0.5-3% Ethoxydiglycol;
0.1-0.5% Sodium Polystyrene Sulfonate;
0.2-10 Dimethyl Sulfone;
0.25-1% Panthenol;
0.1-0.75 Triethyl Citrate;
0.05-0.25 Phylantriol; and
0.1-0.75 Polyacrylamidomethylpropane Sulfonic Acid.

2. The one part solution as defined in claim 1 wherein said composition further comprises 0.5-1.5% fragrance.

3. The one part solution as defined in claim 1 wherein said composition further comprises 0.5-3.0% extracts of Althea Officinalis Root, Chamomilla Recutita (Matricaria) Flower, Citrus Aurantium Dulcis (Orange) Peel, Achillea Millefolium, Foeniculum Vulgare (Fennel) Fruit, Glycyrrhiza Glabra (Licorice) Rhizome/Root, Ginkgo Biloba Leaf.

4. A method of straightening hair comprising the steps of
applying a one part solution to human hair, said one part solution further comprising:
57-92% water;
3.5-8% PEG-8;
2-10% Urea;
0.5-3.0% Polysorbate 20;
0.005-0.5% Methylisothiazolinone;
0.1-0.5% Hydrolyzed Corn Protein;
0.1-0.5% Oat Amino Acids;
0.1-0.5% Hydrolyzed Soy Protein;
0.02-0.1% Disodium EDTA;
0.05-0.50% Hydrolyzed Abalone Protein;
0.5-3% Ethoxydiglycol;
0.1-0.5% Sodium Polystyrene Sulfonate;
0.2-10 Dimethyl Sulfone;
0.25-1% Panthenol;
0.1-0.75 Triethyl Citrate;
0.05-0.25 Phylantriol; and
0.1-0.75 Polyacrylamidomethylpropane Sulfonic Acid;
applying to the hair soaked with said solution a hot iron set a temperature between 200 and 500 degrees Fahrenheit;
with said hot iron, straighten the hair using tension and a comb between one and six times as dictated by the type of hair.

5. The method as defined in claim 4 wherein said one part solution further comprises 0.5-1.5% fragrance.

6. A method of semi-permanent straightening of hair comprising the steps of:
washing the hair with a clarifying shampoo;
drying the hair completely;
applying a one part solution to human hair, said one part solution further comprising:
57-92% water;
3.5-8% PEG-8;
2-10% Urea;
0.5-3.0% Polysorbate 20;
0.005-0.5% Methylisothiazolinone;
0.1-0.5% Hydrolyzed Corn Protein;
0.1-0.5% Oat Amino Acids;
0.1-0.5% Hydrolyzed Soy Protein;
0.02-0.1% Disodium EDTA;
0.05-0.50% Hydrolyzed Abalone Protein;
0.5-3% Ethoxydiglycol;
0.1-0.5% Sodium Polystyrene Sulfonate;
0.2-10 Dimethyl Sulfone;
0.25-1% Panthenol;
0.1-0.75 Triethyl Citrate;
0.05-0.25 Phylantriol; and
0.1-0.75 Polyacrylamidomethylpropane Sulfonic Acid;
drying the hair completely;
straightening the hair with a flat iron at 410 degrees Fahrenheit; and
repeating from one to eight times.

7. The method as defined in claim 6 wherein said one part solution further comprises 0.5-1.5% fragrance.

8. The method as defined in claim 6 wherein said one part solution further comprises 0.5-3.0% extracts of Althea Officinalis Root, Chamomilla Recutita (Matricaria) Flower, Citrus Aurantium Dulcis (Orange) Peel, Achillea Millefolium, Foeniculum Vulgare (Fennel) Fruit, Glycyrrhiza Glabra (Licorice) Rhizome/Root, Ginkgo Biloba Leaf.

9. A method of curling hair comprising the steps of
applying a one part solution to human hair, said one part solution further comprising:
57-92% water;
3.5-8% PEG-8;
2-10% Urea;
0.5-3.0% Polysorbate 20;
0.005-0.5% Methylisothiazolinone;
0.1-0.5% Hydrolyzed Corn Protein;
0.1-0.5% Oat Amino Acids;
0.1-0.5% Hydrolyzed Soy Protein;
0.02-0.1% Disodium EDTA;
0.05-0.50% Hydrolyzed Abalone Protein;
0.5-3% Ethoxydiglycol;
0.1-0.5% Sodium Polystyrene Sulfonate;
0.2-10 Dimethyl Sulfone;
0.25-1% Panthenol;
0.1-0.75 Triethyl Citrate;
0.05-0.25 Phylantriol; and
0.1-0.75 Polyacrylamidomethylpropane Sulfonic Acid;
applying to the hair soaked with said solution a hot curling device set a temperature between 200 and 500 degrees Fahrenheit.

10. The method as defined in claim 9 wherein said one part solution further comprises 0.5-1.5% fragrance.

11. The method as defined in claim 9 wherein said one part solution further comprises 0.5-3.0% extracts of Althea Officinalis Root, Chamomilla Recutita (Matricaria) Flower, Citrus Aurantium Dulcis (Orange) Peel, Achillea Millefolium, Foeniculum Vulgare (Fennel) Fruit, Glycyrrhiza Glabra (Licorice) Rhizome/Root, Ginkgo Biloba Leaf.
